(19) United States
(12) Patent Application Publication
(10) Pub. No.: US 2014/0324067 A1
(43) Pub. Date: Oct. 30, 2014

(54) SUBCUTANEOUS TUNNELING AND IMPLANTATION TOOLS FOR A DISK-SHAPED SENSOR

(71) Applicant: Senseonics, Incorporated, Germantown, MD (US)

(72) Inventors: Jeremy Emken, Germantown, MD (US); Andrew DeHennis, Germantown, MD (US); Phil Huffstetler, Germantown, MD (US); Todd Whitehurst, Germantown, MD (US)

(73) Assignee: SENSEONICS, INCORPORATED, Germantown, MD (US)

(21) Appl. No.: 14/151,452

(22) Filed: Jan. 9, 2014

Related U.S. Application Data
(60) Provisional application No. 61/750,489, filed on Jan. 9, 2013.

(51) Int. Cl. A61B 17/34; A61B 5/00

(52) U.S. Cl. CPC A61B 17/3468; A61B 5/6846
USPC 606/129

(57) ABSTRACT

Tunneling and implantation tools that may be used to implant a sensor within a living animal. For example, the sensor may be implanted in subcutaneous tissue below the skin. The sensor may be disk-shaped and may be capable of being compressed (e.g., wrapped) in a more cylindrical shape. The sensor may or may not be able expand on its own (i.e., to force tissue apart to accommodate the sensor in its full disk-shape) after insertion into the living animal. The tunneling tool may have a flat and wide tip, a vibration device, arms attached to a rod at its tip, a spring in a hollow tube at the end of a rod, and/or oscillating teeth. The implantation tool may have jaws that clamp and then release the sensor and/or rods that insert into channels of the sensor and spread apart to assist in opening up a compressed sensor.

### CROSS-REFERENCE TO RELATED APPLICATIONS

The present application claims the benefit of priority to U.S. Provisional Application Ser. No. 61/750,489, filed on Jan. 9, 2013, which is incorporated herein by reference in its entirety.

### BACKGROUND

1. Field of Invention

The present invention relates generally to tools for implanting a sensor within a living animal. Specifically, the present invention relates to tunneling and implantation tools for implanting a sensor within a living animal.

2. Discussion of the Background

Implantable sensors may be implanted within a living animal (e.g., a human) and may detect the presence or measure the concentration of an analyte (e.g., glucose or oxygen) in a medium (e.g., blood) within the living animal. Some implantable sensors are implanted in subcutaneous tissue below the skin. The subcutaneous insertion of a sensor is a relatively simple procedure and may take less than five minutes.

Known tools for inserting a sensor in subcutaneous tissue include both a tunneling tool and an insertion tool. FIG. 1 illustrates an example of a known tunneling tool 102 and a known implantation tool 104. The tunneling tool 102 and implantation tool 104 may be designed based on the configuration of the sensor 106 that is to be implanted by the tunneling and implantation tools 102 and 104. The sensor 106 may have a small and relatively cylindrical shape.

The implantation of a sensor is not meant to be painful. The tunneling tool 102 may have a rod/shaft 108 extending from a handle 107, wherein the rod 108 may have a dull tip 109 at the end of the rod 108. The rod 108 may be meant to make space for the sensor 106, rather than pierce through vital tissue in the portion of the living animal (e.g., wrist, arm, leg, or abdomen) into which the sensor 106 is inserted.

The sensor 106 may be implanted under local anesthetic. The implantation process may begin with a small incision (e.g., with a #15 blade scalpel). The tunneling tool 102 may then used to create a pocket for the sensor 106. The tunnel may be immediately subcutaneous, e.g., no more than 1-2 cm below the surface of the skin. The pocket may follow the fascial plane at the bottom of the subcutaneous layer.

The implantation tool 104 (i.e., insertion tool or device) may then be used to deliver the sensor 106 to the patient. The implantation tool 104 (i.e., insertion tool or device) may have a rod/shaft 113 that is shaped similarly to the rod 18 of the tunneling tool 102. The implantation tool 104 may contain a compartment that holds the sensor 106 for easy insertion into the living animal (i.e., the patient).

The implantation tool 104 may be inserted in the incision. The implantation device may have a sliding switch 112 that, once the sensor 106 is inserted far enough into the patient (e.g., to a second line 110 on the rod 113 of the insertion device 104), is pulled back in order to release the sensor 106 into a body part (e.g., wrist, arm, leg, or abdomen) of the patient. For example, pulling back the switch 112 may pull back a polytetrafluoroethylene (PTFE) tube around the sensor 106 without moving the sensor 106 itself. The insertion tool 104 may subsequently be removed, leaving the sensor 106 behind in the pocket created by the tunneling tool 102.

As illustrated in FIG. 2, the sensor 106 may be part of a sensor system that also includes an external device 214. The external device 214 may supply power and energy required by the sensor 106 for proper sensor operation. The sensor 106 may be inefficient in terms of energy considerations, and the energy necessities may require that the external device 214 be large to compensate for these inefficiencies.

For example, the sensor 106 may be less efficient due to a non-ideal orientation of the wire inductor coils 216 and 218 of the external device 214 and sensor 106, respectively, as shown in FIG. 2. When the center of the coils 218 lies perpendicular to the coils 216 inside of the external device 214, only a fraction of the possible power transfer is available.

There is presently a need in the art for a more efficient sensor (e.g., a sensor having a coil with a center that, when implanted, lies parallel to the coils inside an external device) and for tools to implant a sensor having a different configuration than sensor 106 (e.g., a more efficient sensor having a coil with a center that, when implanted, lies parallel to the coils inside an external device) in a living animal (e.g., at the bottom of the subcutaneous layer).

### SUMMARY

In one aspect of the invention, a more efficient sensor is provided comprising a sensor having a coil, the center of which would lie parallel to the coils inside an external device. With such a coil, a much larger fraction of the possible power transfer is available than a coil with a center than lies perpendicular to the coil of an external device. However, reorienting the wire inductor coils of a sensor to be parallel (as opposed to perpendicular) to coils of an external device may require that the sensor be slightly larger (i.e., wider) and disk-shaped. Embodiments of the present invention provide tools for inserting a disk-shaped sensor into a living animal (e.g., at the bottom of the subcutaneous layer).

One aspect of the present invention provides a subcutaneous tunneling tool for implanting a disk-shaped sensor. The tunneling tool may include a handle, a rod, and a vibration device. The rod may extend from the handle. The rod may include a tip at the end of the rod. The vibration device may be configured to vibrate the tip. In some embodiments, the tip may be flat and wide.

Another aspect of the present invention provides a subcutaneous tunneling tool for implanting a disk-shaped sensor. The tunneling tool may include a handle, a rod, and an actuator. The rod may extend from the handle. The rod may include a tip at the end of the rod and one or more arms attached to the rod at the tip. The actuator may be configured to force the one or more arms away from the rod. In some embodiments, the one or more arms may be spring-loaded. In some embodiments, the actuator may be a slider.

Yet another aspect of the present invention provides a subcutaneous tunneling tool for implanting a disk-shaped sensor. The tunneling tool may include a handle, a rod, and an actuator. The rod may extend from the handle. The rod may include a hollow tube at the end of the rod and a spring that, when confined in tension in the hollow tube, is compressed and, when relieved of tension out of the hollow tub, fans open. The actuator may be configured to force the spring out of the hollow tube and relieve the spring of tension. In some embodiments, the actuator may be a slider.

Still another aspect of the present invention is a subcutaneous tunneling tool for implanting a disk-shaped sensor. The tunneling tool may include a handle and a rod. The rod may extend from the handle. The rod may include a tip at the end of the rod and teeth configured to oscillate in and out of the rod when actuated.

Another aspect of the present invention provides a subcutaneous tunneling tool for implanting a disk-shaped sensor. The tunneling tool may include a jaws, a spring, and an actuator. The jaws may be configured to clamp the sensor, which has been wrapped up into a cylindrical shape, and keep the sensor wrapped up in the cylindrical shape. The spring may be configured to keep the jaws clamped together via tension of the spring. The actuator configured to release the tension of the spring and release the sensor from the jaws.

A further aspect of the present invention provides a subcutaneous implantation tool for implanting a disk-shaped sensor. The implantation tool may include a first rod configured for insertion into a first channel in the sensor, a second rod configured for insertion into a second channel in the sensor, and an actuator configured to spread apart the first and second rods apart such that the sensor, which has the first and second ends connected to the first and second rods, respectively, is unwrapped. In one embodiment, the first and second channels may be tapered.

Yet another aspect of the invention provides a subcutaneous tunneling tool for implanting a disk-shaped sensor. The tunneling tool may include a handle and a rod extending from the handle. The rod may include a flat and wide tip at the end of the rod.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting embodiments of the present invention. In the drawings, like reference numbers indicate identical or functionally similar elements.

FIG. 1 is a perspective view of an example of known tunneling and implantation tools.

FIG. 2 illustrates an orientation of wire inductive coils of a known sensor system.

FIG. 3 illustrates the improved orientation between inductive elements of a sensor system embodying aspects of the present invention.

FIG. 4 illustrates the tip of a tunneling tool having a duck bill design embodying aspects of the present invention.

FIG. 5 illustrates a tunneling tool having a vibration design embodying aspects of the present invention.

FIG. 6 illustrates a tunneling tool having an arm design embodying aspects of the present invention.

FIG. 7 illustrates a tunneling tool having a fan design embodying aspects of the present invention.

FIG. 8 illustrates a tunneling tool having a teeth design embodying aspects of the present invention.

FIG. 9 illustrates an implantation tool having a jaws design embodying aspects of the present invention.

FIGS. 10(a) through 10(e) illustrate an implantation tool having a rod design embodying aspects of the present invention.

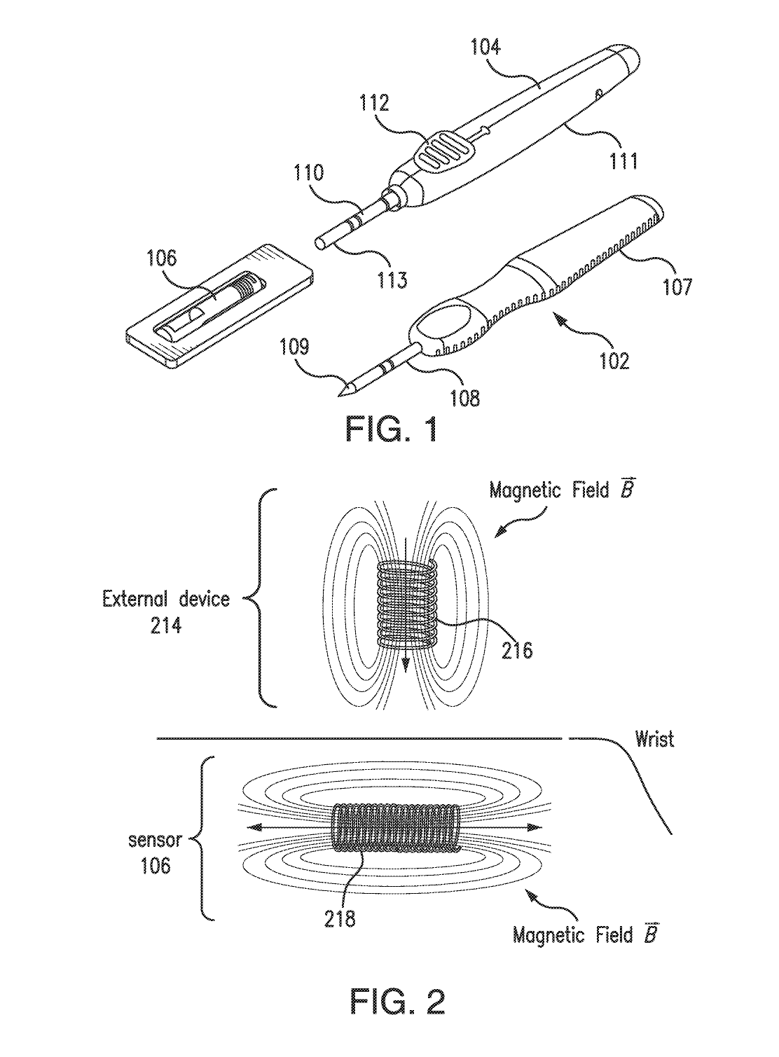

### DETAILED DESCRIPTION OF PREFERRED EMBODIMENTS

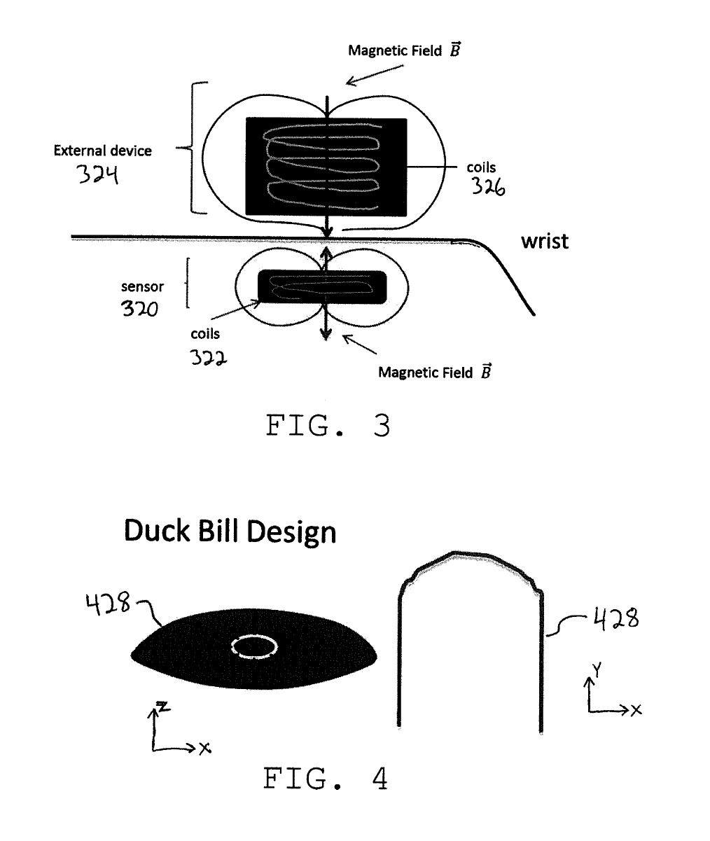

FIG. 3 illustrates the orientation between inductive elements 322 and 326 of a sensor system embodying aspects of the present invention. As shown in FIG. 3, in some non-limiting embodiments, the system includes a sensor 320 and an external sensor reader/device 324. The sensor 320 may be implanted in a living animal (e.g., a living human). The sensor 320 may be implanted, for example, in a living animal's arm, wrist, leg, abdomen, or other region of the living animal suitable for sensor implantation. For example, as shown in FIG. 3, in one non-limiting embodiment, the sensor 320 may be implanted in or adjacent to the subcutaneous layer below the skin of a patient's wrist (e.g., no more than approximately 1-2 cm below the surface of the skin).

The external device 324 may supply power and energy required by the sensor 320 for proper sensor operation. The sensor 320 may convey measurement results and other information to the external device 324.

The sensor 320 may have one or more coils 322 with a center that lies parallel to one or more coils 326 inside an external device 324. With such a coil, a much larger fraction of the possible power transfer is available than the possible power transfer available with a coil (e.g., coil(s) 218 of FIG. 2) having a center than lies perpendicular to the one or more coils 326 of the external device 324. Thus, in some embodiments, the sensor 320 may increase power transfer between the external device 324 and implanted sensor 320.

However, the parallel (as opposed to perpendicular) orientation of the one or more coils 322 of the sensor 320 relative to the one or more coils 326 of external device 324 may require the sensor 320 be slightly larger (e.g., a wider sensor by a few mm) and disk-shaped but allow the external power device 324 to be smaller (e.g., relative to external device 214). New tunneling and implantation tools may be required to insert a disk-shaped sensor 320, which may be larger (i.e., wider) than existing sensor 106.

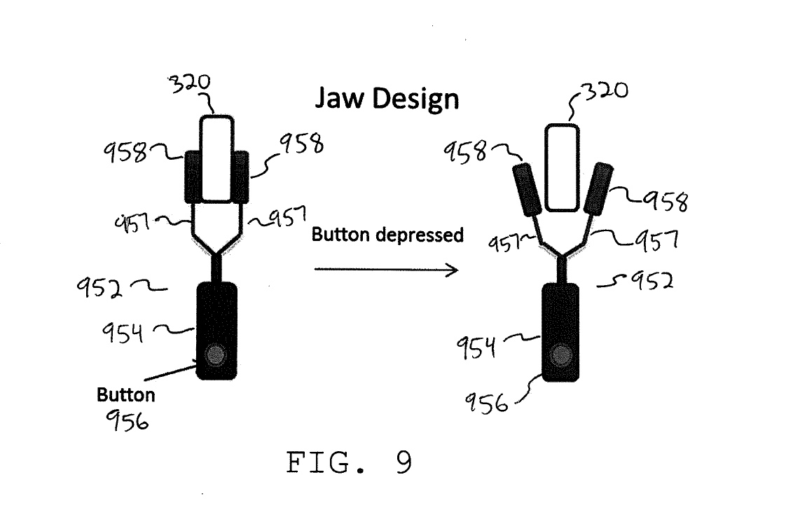

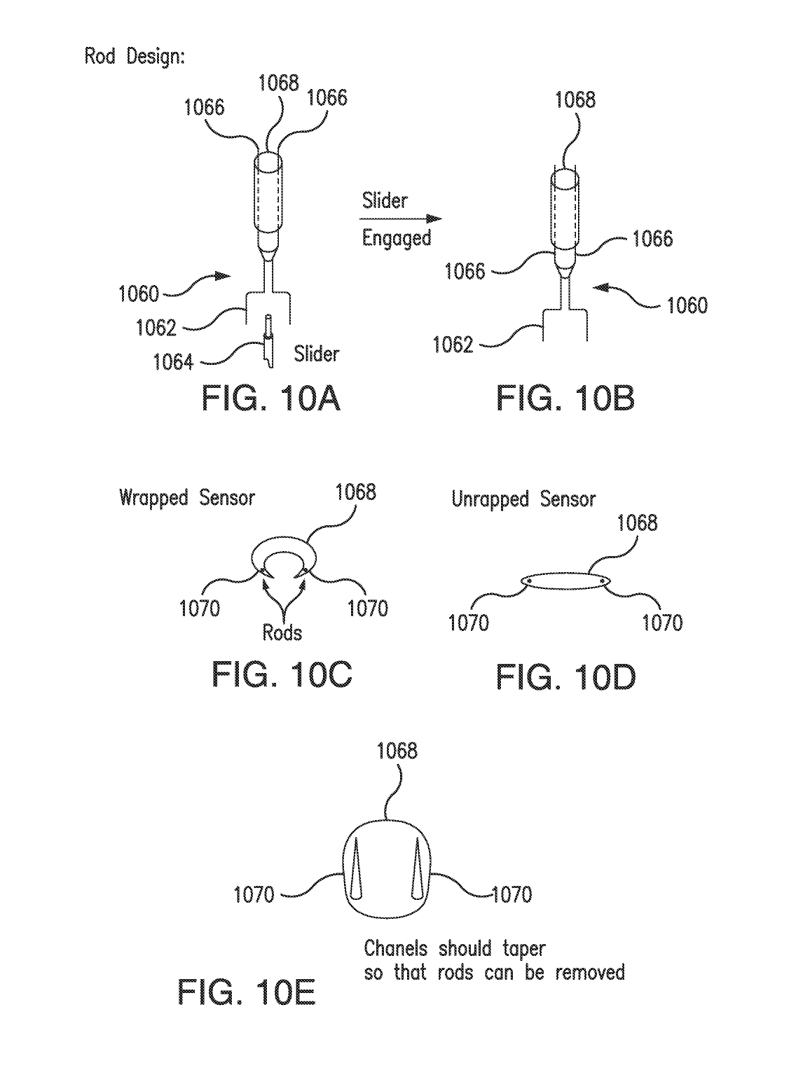

In some embodiments, the disk-shaped sensor 320 may be compressed (e.g., wrapped into a more cylindrical shape) for implantation. For example, FIGS. 10(a)-10(e) illustrate a non-limiting embodiment of a disk-shaped sensor 1068 that may be compressed from an unwrapped state (see FIGS. 10(b), 10(d), and 10(e)) into a wrapped state (see FIGS. 10(a) and 10(c)). The disk-shaped sensor 1068 may have a more cylindrical shape in the wrapped state than in the unwrapped state.

In some embodiments where the sensor 320 may be compressed, the sensor 320 may be able to open up/expand on its own within a slit from the compressed cylindrical shape into a disk shape after implantation into the slit. That is the sensor 320 may be capable of springing open and creating a pocket subcutaneously for itself (e.g., by pushing bodily tissue out of its way). However, in other embodiments where the sensor 320 may be compressed, the sensor 320 may not be capable of forcing tissue apart to accommodate the sensor 320 in its full disk-shape. In some alternative embodiments, the sensor 320 may not be capable of being compressed into a more cylindrical shape for implantation.

Whether tunneling and implantation tools different than the existing tunneling and implantation tools 102 and 104 may be required for use with the sensor 320 may depend on whether the disk-shaped sensor 320 is capable being compressed for implantation and on whether the sensor 320, if capable of being compressed, is capable of opening up/expanding on its own after subcutaneous implantation. For instance, on one hand, if the sensor 320 is capable of being compressed into a more cylindrical shape from its original disk shape for subcutaneous implantation and then, after implantation, opens up to its required disk shape, then only minor changes to the existing tunneling and implantation tools 102 and 104 may be required. On the other hand, if the sensor 320 is not capable of being compressed or not capable of opening up/expanding on its own from a compressed shape to its disk shape after implantation, new tunneling and/or implantation tools may be required.

In regard to tunneling tools, a tunneling tool in accordance with one non-limiting embodiment of the present invention may be similar to existing tunneling tool 102 (i.e., having a cylindrical rod with a blunt tip at the end), but have a cylindrical rod with a larger diameter than the compressed sensor 320 to allow the sensor 320 some room to open may be used with a disk-shaped sensor 320 that is capable of being wrapped up into a cylindrical shape and capable of forcing tissue apart to accommodate the sensor 320 in its full disk-shape. However, this tunneling tool could not be used if the disk-shaped sensor 320 (a) cannot be compressed or (b) can be wrapped up into a cylindrical shape but cannot force tissue apart to accommodate the sensor 320 in its full disk-shape.

In contrast, the tunneling tools in accordance with the embodiments of the present invention may be used with a disk-shaped sensor 320 cannot be wrapped up into a cylindrical shape or can be compressed but cannot force tissue apart to accommodate it in its full disk-shape.

As illustrated in FIG. 4, a tunneling tool/device in accordance with some embodiments of the present invention may have a tip 428 that is less cylindrical than the tip 109 of existing tunneling device 102. For example, the tip 428 may be flatter and wider (i.e., shaped like a duck bill) and, as a result, may clear more tissue apart when used before implantation.

In some embodiments, the tunneling tool having tip 428 may also have a handle and rod that may be similar to those of existing tunneling tool 102. In one non-limiting embodiment, the tunneling tool may be a wider and flatter version of the existing tunneling tool 102.

In some embodiments, the tunneling tool having a wide and flat shape creates a larger pocket underneath the skin so that even a disk-shape sensor that cannot force bodily tissue aside on its own may, nonetheless, opens up in the pocket created by the wide and flat tip.

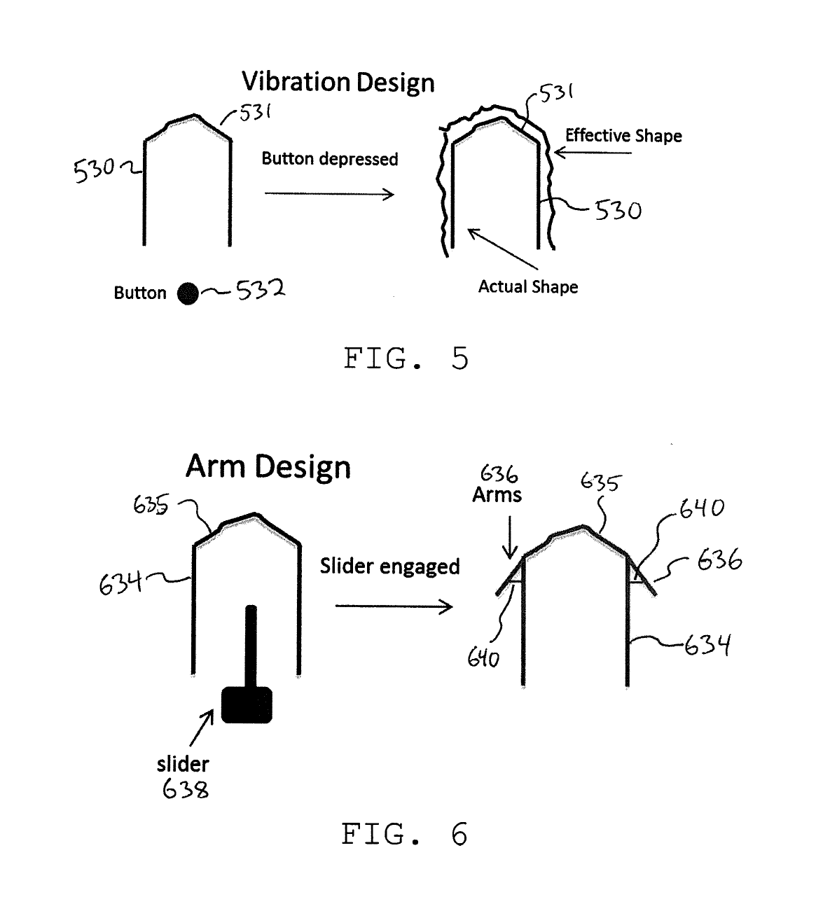

As illustrated in FIG. 5, a tunneling tool/device in accordance with some embodiments of the present invention may have a rod 530 and a tip 531 at the end of the rod 530. The tunneling tool may have a vibration device/element (e.g., a vibrating head) that may be capable of vibrating (e.g., gently vibrating) the tip 531 and/or the rod 530. The rod 530 may extend from a handle that may be similar to the handle 107 of existing tunneling tool 102. However, the handle may have an actuator 532 (e.g., a button or switch) that, when actuated (e.g., depressed), causes the vibration device to vibrate the tip 531 and/or the rod 530. In one non-limiting embodiment, the vibration device is located in the rod 530, but, in alternative embodiments, the vibration device may be located elsewhere (e.g., in the handle).

The vibrations of the tip 531 may effectively make a tunnel that is larger than the actual tip 531. Thus, the vibration device may effectively increase the size of the tip 531 of the tunneling tool. Further, the vibrations may enable the tunneling tool to create a pocket wider than the incision in the skin.

In a non-limiting embodiment, the tip 531 and/or rod 530 may be flat and wide (i.e., similar to tip 428 of FIG. 4). However, this is not necessary, and, in an alternative embodiment, the tip 531 and rod 530 may have a cylindrical geometry similar to that of the tip 109 and rod 108 of existing tunneling tool 107.

As illustrated in FIG. 6, a tunneling tool/device in accordance with some embodiments of the present invention may have a rod 634, a tip 635 at the end of the rod 634, and one or more arms 636 attached (e.g., pivotally attached) to the rod 634 at the tip 635. The rod 634 may extend from a handle that may be similar to the handle 107 of existing tunneling tool 102. However, the handle may have an actuator 538 (e.g., a slider or button) that, when actuated (e.g., engaged, pushed, or depressed), forces the one or more arms 636 away from the rod 634.

The arms 636 may effectively increase the size of the tip 635 of the tunneling tool. As the one or more arms 636 would clear away more tissue, a tunneling tool having one or more arms 636 may require a smaller incision than tunneling tool that does not have arms to clear away tissue. Further, the arms 636 may enable the tunneling tool to create a pocket wider than the incision in the skin.

In some non-limiting embodiments, the arms 636 may be spring-loaded. For example, in one non-limiting embodiment, the arms may be spring-loaded by one or more springs 640.

In some non-limiting embodiments, the rod 634 may have a cylindrical shape similar to that of rod 108 of existing tunneling tool 102. However, this is not required, and alternative embodiments may have arms attached to a rod have a flat and wide shape.

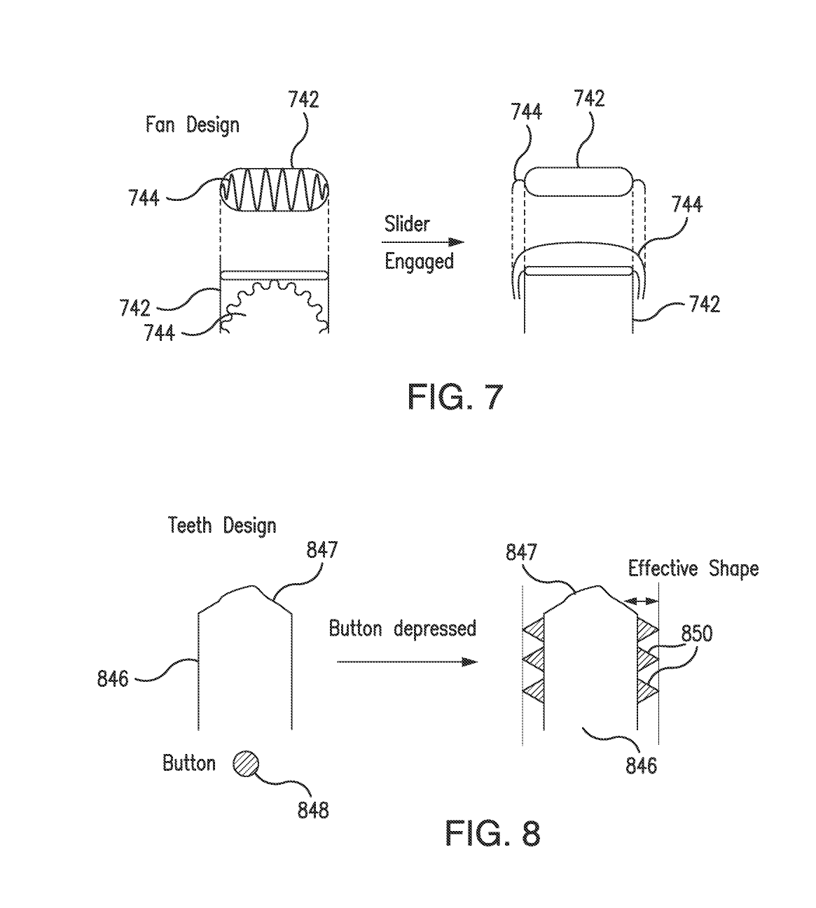

As illustrated in FIG. 7, a tunneling tool/device in accordance with some embodiments of the present invention may have a rod that includes a hollow tube 742 at the end of the rod and a spring 744 that may act as the tip of the tunneling tool. In one non-limiting embodiment, spring 744 may be a compression spring. Spring 744 may be capable of being compressed to fit within the hollow tube 742. When in the hollow tube 742, the spring 744 may be confined under tension within the hollow tube 742 in its compressed state. Spring 742 may be capable of fanning open/expanding when relieved of tension (e.g., when pushed out of the hollow tube 742 and no longer confined thereby). The rod may extend from a handle that may be similar to the handle 107 of existing tunneling tool 102. However, the handle may have an actuator (e.g., a slider) that, when actuated (e.g., engaged, pushed, or depressed), pushes the spring 744 forward and out of the hollow tube 742, which may lead to the spring 744 fanning open/expanding. In one non-limiting embodiment, the actuator may be a slider, and the spring 744 may be attached to end of the slider. In some embodiments, it may be possible to retract the spring 744 back into the hollow tube 742 by pulling slider back.

Thus, instead of a solid, blunt tip, the tunneling tool may have a tip that is capable of fanning/springing open. In one non-limiting embodiment, the actuator may be a slider that, when engaged, thrusts the fanned tip forwards and, once the tip is free of the side confines of the hollow tube 742, the tip expands due to the restorative force of the compressed spring 744.

The spring 744 may effectively increase the size of the tip of the tunneling tool and the size of the tunnel created thereby. As the expansion of the spring 744 would clear away tissue, the tunneling tool having spring 744 may require a smaller incision than tunneling tool that does not have arms to clear away tissue. Further, the spring 744 may enable the tunneling tool to create a pocket wider than the incision in the skin.

As illustrated in FIG. 8, a tunneling tool/device in accordance with some embodiments of the present invention may have a rod 846, a tip 847 at the end of the rod 846, and one or more teeth 850. The teeth 850 may be in the proximity of the tip 847 and may oscillate in and out of the rod 846 when actuated. The rod 846 may extend from a handle that may be similar to the handle 107 of existing tunneling tool 102. However, the handle may have an actuator 848 (e.g., a button or switch) that, when actuated (e.g., depressed), causes the teeth 850 to oscillate in and out of the rod 846. In use, the actuator 848 may be actuated as the tool is tunneling, which may cause the teeth to extend from and retract into the rod 846. In one non-limiting embodiment, the tunneling tool having teeth 850 may be electrically powered.

Oscillation of the teeth 850 may effectively make a tunnel that is larger than the actual tip 847. Thus, the teeth 850 may effectively increase the size of the tip 847 of the tunneling tool. Further, oscillation of the teeth 850 may enable the tunneling tool to create a pocket wider than the incision in the skin.

In regard to implantation tools, an implantation tool in accordance with one non-limiting embodiments of the present invention may be similar to existing implantation tool 104 (i.e., having a cylindrical shape with a compartment to house the sensor until the tool is pushed sufficiently far into the incision, at which point a slider is engaged that releases the sensor), but may have a larger design to accommodate the larger sensor 320. However, this implantation tool could only be used if the disk-shaped sensor 320 can be wrapped up into a cylindrical shape and is capable of forcing tissue apart to accommodate the sensor 320 in its full disk-shape.

As illustrated in FIG. 9, an implantation tool/device 952 in accordance with some embodiments of the present invention may have a handle 954 and jaws 957. The jaws 957 may be configured to clamp a disk-shaped sensor 320, which has been wrapped up into a cylindrical shape, and keep the sensor 320 wrapped up in the cylindrical shape. The handle 954 may include an actuator 956 (e.g., a button or switch) that, when actuated (e.g., depressed), causes the jaws 957 to release the sensor 320. In some non-limiting embodiments, the jaws 957 may include gripping portions 958 configured to grip the sensor 320 when clamped by the jaws 957.

In some non-limiting embodiments, the implantation tool 952 may include a spring configured to keep the jaws clamped together via tension of the spring. In these embodiments, the actuator 956 may be configured to release the sensor 320 from the jaws 957 by releasing the tension of the spring.

Although implantation tool 952 may be used to implant a disk-shaped sensor 320 that has been wrapped up in the cylindrical shape, in some embodiments, implantation tool 952 may additionally or alternatively be used to implant a cylindrical sensor, such as the existing sensor 106.

As illustrated in FIGS. 10(a)-10(e), an implantation tool/device 1060 in accordance with some embodiments of the present invention may be configured to implant a disk-shaped sensor 1068 having channels 1070 (e.g., first and second channels). In some embodiments, the disk-shaped sensor 1068 may be able to be wrapped into a more cylindrical shape (see FIG. 10(c)) but may not be able to force tissue apart to accommodate the full disk-shape of the sensor 1068 in its unwrapped state (see FIG. 10(d)).

The implantation tool 1060 may have a handle 1062 and rods 1066 (e.g., first and second rods). The rods 1066 may be configured to be inserted into respective channels 1070 of the sensor 1068. The handle 1062 may have an actuator 1064 (e.g., a slider, button, or switch) that may be configured to spread apart the rods 1066. As the rods 1066 are spread apart, the rods 1066 would spread apart/unwrap the sensor 1068 into which the rods 1066 are inserted. In this way, the spreading apart of the rods 1066 would force tissue apart to accommodate the sensor 1068 in its unwrapped state.

The rods 1066 may be configured to be removed from the channels 1070 of the sensor 1068 after the sensor 1068 is spread apart. In some non-limiting embodiments, the channels 1070 may be at respective ends of the sensor 1068. In some non-limiting embodiments, the channels 1070 of the sensor 1068 may be tapered.

The tunneling and implantation tools described above with respect to embodiments of the present invention may be used for insertion of disk-shaped sensors that allow for more efficient power transfer between the sensor and an external power source and, thus, allowing for the external source to be smaller.

In some embodiments, a tunneling tool in accordance with an above-described tunneling tool embodiment of the present invention may be used in combination with an implantation tool in accordance with an above-described implantation tool embodiment of the present invention. However, this is not required, and, in other embodiments, a tunneling tool in accordance with an above-described tunneling tool embodiment of the present invention may be used with an existing implantation tool or an existing tunneling tool may be used with an implantation tool in accordance with an above-described implantation tool embodiment of the present invention.

The tunneling and implantation tools in accordance with the above-described embodiments of the present invention may be implanted by a physician but implantation by patient (e.g., at home) is also an option. For example, in some non-limiting embodiments, one or more of the tunneling and implantation tools in accordance with the above-described embodiments of the present invention may be included in an at-home sensor insertion kit, which may be available from physicians so that patients can easily implant a sensor in the comfort of their own home.

Embodiments of the present invention have been fully described above with reference to the drawing figures. Although the invention has been described based upon these preferred embodiments, it would be apparent to those of skill in the art that certain modifications, variations, and alternative constructions could be made to the described embodiments within the spirit and scope of the invention.

What is claimed is:

1. A subcutaneous tunneling tool for implanting a disk-shaped sensor, the tunneling tool comprising:
a handle;
a rod extending from the handle, wherein the rod includes a tip at the end of the rod; and
a vibration device configured to vibrate the tip.

2. The tunneling tool of claim 1, wherein the tip is flat and wide.

3. A subcutaneous tunneling tool for implanting a disk-shaped sensor, the tunneling tool comprising:
a handle;
a rod extending from the handle, wherein the rod includes:
a tip at the end of the rod; and
one or more arms attached to the rod at the tip; and
an actuator configured to force the one or more arms away from the rod.

4. The tunneling tool of claim 3, wherein the one or more arms are spring-loaded.

5. The tunneling tool of claim 3, wherein the actuator is a slider.

6. A subcutaneous tunneling tool for implanting a disk-shaped sensor, the tunneling tool comprising:
a handle;
a rod extending from the handle, wherein the rod includes:
a hollow tube at the end of the rod; and
a spring that, when confined in tension in the hollow tube, is compressed and, when relieved of tension out of the hollow tub, fans open; and
an actuator configured to force the spring out of the hollow tube and relieve the spring of tension.

7. The tunneling tool of claim 6, wherein the actuator is a slider.

8. A subcutaneous tunneling tool for implanting a disk-shaped sensor, the tunneling tool comprising:
a handle;
a rod extending from the handle, wherein the rod includes:
a tip at the end of the rod; and
teeth configured to oscillate in and out of the rod when actuated.

9. The tunneling tool of claim 8, wherein the teeth, when actuated, extend from the rod and retract into the rod.

10. A subcutaneous implantation tool for implanting a disk-shaped sensor, the implantation tool comprising:
jaws configured to clamp the sensor, which has been wrapped up into a cylindrical shape, and to keep the sensor wrapped up in the cylindrical shape;
a spring configured to keep the jaws clamped together via tension of the spring;
an actuator configured to release the tension of the spring and release the sensor from the jaws.

11. The implantation tool of claim 10, further comprising first and second gripping portions configured to grip the sensor when clamped by the jaws.

12. A subcutaneous implantation tool for implanting a disk-shaped sensor, the implantation tool comprising:
a first rod configured for insertion into a first channel in the sensor;
a second rod configured for insertion into a second channel in the sensor; and
an actuator configured to spread apart the first and second rods apart such that the sensor, which has the first and second ends connected to the first and second rods, respectively, is unwrapped.

13. The implantation tool of claim 12, wherein the first and second rods are configured to be removed from the first and second channels, respectively, of the sensor after being spread apart.

14. The implantation tool of claim 12, wherein the first channel is at a first end of the sensor, and the second channel is at a second end of the sensor.

15. The implantation tool of claim 12, wherein the first and second channels are tapered.

16. A subcutaneous tunneling tool for implanting a disk-shaped sensor, the tunneling tool comprising:
a handle;
a rod extending from the handle, wherein the rod includes a flat and wide tip at the end of the rod.
